(19) United States
(12) Patent Application Publication
(10) Pub. No.: US 2019/0216430 A1
(43) Pub. Date: Jul. 18, 2019

(54) SYSTEM AND METHOD FOR ULTRASOUND FLOW IMAGING

(71) Applicant: GENERAL ELECTRIC COMPANY, Schenectady, NY (US)

(72) Inventor: Ralph Thomas Hoctor, Saratoga Springs, NY (US)

(21) Appl. No.: 15/871,090

(22) Filed: Jan. 15, 2018

(51) Int. Cl. A61B 8/00; A61B 8/08; G01S 15/89; A61B 8/06

(52) U.S. Cl. CPC A61B 8/461; A61B 8/488; G01S 15/8979; G01S 15/8927; A61B 8/06; A61B 8/5207

(57) ABSTRACT

A method for ultrasound flow imaging includes transmitting a set of transmission beams through a region of interest having a plurality of spatial locations. The method further includes generating demodulated data in response to the set of transmission beams. Further, the method includes obtaining a plurality of wave-number vectors and location response data corresponding to each spatial location. Moreover, the method includes determining a plurality of Doppler frequency values based on the location response data and determining a flow vector for each spatial location based on the plurality of Doppler frequency values and the plurality of wave-number vectors. The method also includes generating a flow vector image based on the flow vectors corresponding to the plurality of spatial locations within the region of interest, where the flow vector image is representative of a magnitude and direction of blood flow in the region of interest.

### BACKGROUND

Embodiments of the present specification relate generally to ultrasound imaging, and more particularly to systems and methods for generating a flow vector image representative of flow information at a plurality of spatial locations in a region of an object.

Conventional ultrasound imaging systems include an array of ultrasonic transducers which are used to transmit an ultrasound beam (transmitter mode) towards an object and receive a reflected beam from the object being studied. For ultrasound imaging, the array typically has a multiplicity of transducers arranged in a line and driven with separate voltages. The time delay (or phase) and amplitude of the applied voltages may be selected to control the individual transducers to produce ultrasound. These ultrasound waves are combined to form a net ultrasonic wave that travels along a preferred beam direction and is focused at a selected point along the beam. Additionally, by changing the time delay and the amplitude of the applied voltages, the focused and steered beam can be scanned in a plane to image the object.

Similarly, when the transducer is employed to receive the reflected waves (receiver mode), the voltages produced at the receiving transducers are summed so that the net signal is indicative of the ultrasound waves reflected from a single focal point on the object. As with the transmission mode, this focused reception of the ultrasonic energy is achieved by imparting separate time delay (and/or phase shifts) and gains to the signal from each receiving transducer. This form of ultrasonic imaging is referred to as “phased array sector scanning.” This scanning includes a series of measurements in which the steered ultrasound wave is transmitted. The system is then switched to the receive mode after a short time interval and the reflected ultrasonic wave is received and stored. Typically, transmission and reception are steered in the same direction during each measurement to acquire data from a series of points along an acoustic beam or scan line. The receiver is dynamically focused at a succession of ranges along the scan line as the reflected ultrasonic waves are received.

In multi-line acquisition, transducer elements are used to transmit a broad beam that may be several times wider than a single focused transmit beam. Transmitting a broad ultrasonic beam during a single transmit event increases the imaging frame rate by simultaneously generating multiple reflected ultrasound waves within the insonified region.

Typically, reflected waves are processed further either to provide a grey scale image representative of structural information or a color-coded flow image representative of flow information in an organ of interest. Conventional color flow imaging is limited by dependency of a beam-flow angle on the vasculature. Such a dependency poses additional responsibility on the sonographers to interpret the color flow image for flow directions. Vector flow imaging that provides quantitative mapping of blood flow velocities independent of the beam-flow angle, has been proposed in recent years. Vector flow techniques based on triangulation work well for arrays having a large foot-print. However, for arrays having a smaller foot-print, phased array probes and creation of sub-apertures having adequate angle separation may be difficult. Techniques such as blood speckle tracking may provide better flow vector estimates, but requires ultrafast, broad beam acquisition to cover the field of view.

### BRIEF DESCRIPTION

In accordance with one aspect of the present specification, a method for ultrasound flow imaging is disclosed. The method includes transmitting, by a transmitter array, a set of transmission beams through a region of interest having a plurality of spatial locations. The set of transmission beams includes a plurality of transmission beams corresponding to each of a plurality of transmit beam directions selected from a color flow scan sequence. Moreover, the method includes generating, by a receiver array, demodulated data in response to the set of transmission beams. The demodulated data includes a plurality of beam ensemble data sets corresponding to each of the plurality of transmit beam directions, and each of the plurality of beam ensemble data sets includes a plurality of echo signal data sets corresponding to each of the plurality of transmission beams. The method also includes obtaining a plurality of wave-number vectors and location response data corresponding to each spatial location. The location response data includes a subset of the plurality of beam ensemble data sets corresponding to a subset of the plurality of transmit beam directions. Furthermore, the method includes determining a plurality of Doppler frequency values based on the location response data. In addition, the method includes determining a flow vector for each spatial location based on the plurality of Doppler frequency values and the plurality of wave-number vectors. The method also includes generating a flow vector image based on the flow vectors corresponding to the plurality of spatial locations within the region of interest. The flow vector image is representative of a magnitude and direction of blood flow in the region of interest. A non-transitory computer readable medium configured to perform the method for ultrasound flow imaging is also disclosed.

In accordance with another aspect of the present specification, a system for ultrasound flow imaging is disclosed. The system includes a system front-end unit configured to transmit a set of transmission beams through a region of interest having a plurality having a plurality of spatial locations. The set of transmission beams includes a plurality of transmission beams corresponding to each of a plurality of transmit beam directions selected from a color flow scan sequence. The system front-end unit is further configured to generate demodulated data in response to the set of transmission beams. The demodulated data includes a plurality of beam ensemble data sets corresponding to each of the plurality of transmit beam directions and each of the plurality of beam ensemble data sets includes a plurality of echo signal data sets corresponding to each of the plurality of transmission beams. The system further includes a digital processor unit communicatively coupled to the system front-end unit and configured to acquire the demodulated data. The digital processor unit is further configured to obtain a plurality of wave-number vectors and location response data corresponding to each spatial location. The location response data includes a subset of the plurality of beam ensemble data sets corresponding to a subset of the plurality of transmit beam directions. Moreover, the digital processor unit is configured to determine a plurality of Doppler frequency values based on the location response data. The digital processor unit is further configured to determine a flow vector for each spatial location based on the plurality of Doppler frequency values and the plurality of wave-number vectors. Additionally, the digital processor unit is configured to determine a flow vector image based on the flow vectors corresponding to the plurality of spatial locations in the region of interest. The flow vector image is representative of a magnitude and direction of blood flow in the region of interest. The system also includes a display device communicatively coupled to the digital processor unit and configured to display the flow vector image.

In accordance with another aspect of the present specification, a non-transitory computer readable storage medium for ultrasound flow imaging using a processing unit is disclosed. The non-transitory computer readable storage medium includes instructions to command the processor to transmit, by a transmitter array, a set of transmission beams through a region of interest comprising a plurality of spatial locations. The set of transmission beams includes a plurality of transmission beams corresponding to each of a plurality of transmit beam directions selected from a color flow scan sequence. The non-transitory computer readable storage medium includes additional instructions to command the processor to generate, by a receiver array, demodulated data in response to the set of transmission beams. The demodulated data includes a plurality of beam ensemble data sets corresponding to each of the plurality of transmit beam directions and each of the plurality of beam ensemble data sets includes a plurality of echo signal data sets corresponding to each of the plurality of transmission beams. The non-transitory computer readable storage medium includes further instructions to command the processor to obtain a plurality of wave-number vectors and location response data corresponding to each spatial location. The location response data includes a subset of the plurality of beam ensemble data sets corresponding to a subset of the plurality of transmit beam directions. The non-transitory computer readable storage medium includes additional instructions to command the processor to determine a plurality of Doppler frequency values based on the location response data. Further, the non-transitory computer readable storage medium includes instructions to command the processor to determine a flow vector for each spatial location based on the plurality of Doppler frequency values and the plurality of wave-number vectors. The non-transitory computer readable storage medium includes additional instructions to command the processor to generate a flow vector image based on the flow vectors corresponding to the plurality of spatial locations within the region of interest. The flow vector image is representative of a magnitude and direction of blood flow in the region of interest.

### DRAWINGS

These and other features and aspects of embodiments of the present invention will become better understood when the following detailed description is read with reference to the accompanying drawings in which like characters represent like parts throughout the drawings, wherein:

FIG. 1 is a diagrammatic illustration of an ultrasound flow imaging system, in accordance with aspects of the present specification;

FIG. 2 is a schematic representation of data storage in memory, in accordance with aspects of the present specification;

FIG. 3 is an illustration of a wave-number vector at a spatial location, in accordance with aspects of the present specification; and

FIG. 4 is a flow chart of a method for ultrasound flow imaging, in accordance with aspects of the present specification.

### DETAILED DESCRIPTION

As will be described in detail hereinafter, systems and methods for ultrasound imaging are presented. More particularly, the systems and methods are configured for generation of a flow vector image representative of flow information at a plurality of locations in a region of interest.

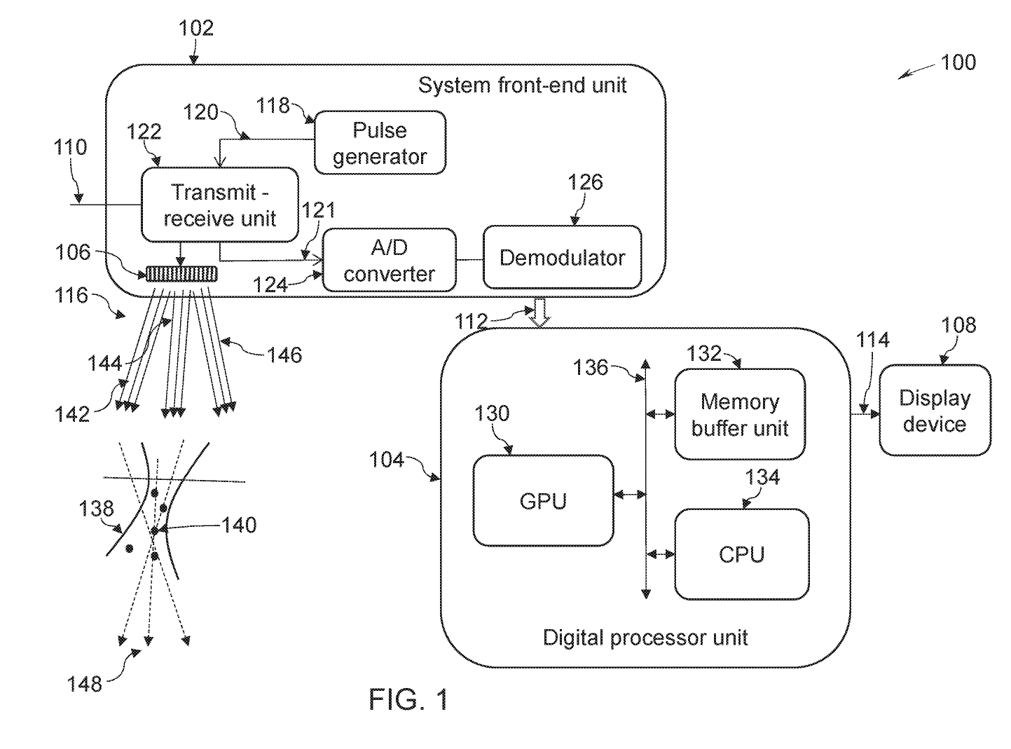

FIG. 1 illustrates a system 100 for ultrasound flow imaging, in accordance with aspects of the present specification. The system 100 is configured to interrogate a region of interest 138 using a color flow scan sequence. The region of interest 138 may include an artery carrying blood, for example. The region of interest 138 includes a plurality of spatial locations 140. In one embodiment, the color flow scan sequence refers to a scanning scheme that defines an ultrasound beam emission sequence used for ultrasound imaging.

Further, the system 100 is configured to receive reflected echo signals generated during the scanning. The system 100 is further configured to process the received echo signals and generate a flow vector image 114, representative of flow information at the plurality of spatial locations 140 in the region of interest 138. The flow information at each of the plurality of spatial locations 140 includes a magnitude and a direction of blood flow.

In a presently contemplated configuration, the system 100 includes a system front-end unit 102 configured to facilitate scanning an object of interest such as a patient, a digital processor unit (DPU) 104, and a display device 108. Also, in one embodiment, the DPU 104 includes a graphics processing unit (GPU) 130, a central processing unit (CPU) 134, and a memory buffer unit 132.

In one embodiment, the system front-end unit 102 includes a pulse generator 118 configured to generate a transmit pulse 120. The system front-end unit 102 further includes a transmit-receive unit 122 communicatively coupled to the pulse generator 118 and configured to generate an ultrasound beam based on a plurality of transmit pulses separated by a pulse repetition interval. The system front-end unit 102 also includes an array of transducer elements 106 communicatively coupled to the transmit-receive unit 122 and configured to operate both in a transmit mode and a receive mode. In the transmit mode, the array of transducer elements 106 is configured to transmit a set of transmission beams towards the region of interest 138. The set of transmission beams includes a plurality of transmission beams 116 transmitted in a plurality of transmit beam directions 142, 144, 146.

The term ‘transmission beam’ as used herein refers to an ultrasound beam transmitted towards the region of interest 138. The plurality of transmission beams 116 is transmitted along one or more transmit beam directions among the plurality of transmit beam directions 142, 144, 146. Also, the term ‘set of transmission beams’ as used herein refers to all the transmission beams corresponding to the plurality of transmit beam directions used for performing a scanning procedure. The term ‘color flow scan sequence’ as used herein refers to a scanning scheme based on the set of transmission beams, the plurality of transmit beam directions, and order of transmissions. Specifically, the color flow scan sequence specifies a plurality of scanning parameters required for an effective scanning. The plurality of scanning parameters includes, but is not limited to, a number of transmit beam directions represented as N, a number of transmission beams for each transmit direction represented as M, a pulse repetition interval (PRI), and a plurality of transmit focal distance values. It may be noted that the set of transmission beams includes a plurality of sets of transmission beams such as the transmission beams 116 transmitted along the plurality of transmit beam directions 142, 144, 146. In the example of FIG. 1, the system front-end unit 102 is depicted as transmitting three sets of transmission beams 116 along each of the plurality of transmit beam directions 142, 144, 146. The system front-end unit 102 is configured to transmit the three sets of transmission beams 116 in quick succession with a time separation specified by a PRI parameter. In general, the system front-end unit 102 is configured to transmit the plurality of sets of transmission beams along the plurality of transmit beam directions 142, 144, 146 sequentially in time.

Upon impinging on the region of interest 138, each transmission beam 116 is reflected from the region of interest 138 towards the system front-end unit 102 that includes the array of transducer elements 106. In the receive mode, the array of transducer elements 106 is configured to measure (sense) the plurality of reflected echo signals generated during the scanning. Within the system front-end unit 102, the transmit-receive unit 122 is configured to receive the echo signals generated during the scanning. The term ‘echo signal’ as used herein refers to the reflected transmission beam. The system front-end unit 102 also includes an analog to digital converter (ADC) 124 and a demodulator 126 for processing the received echo signals.

Furthermore, the system front-end unit 102 is configured to generate demodulated data 112 in response to the sets of transmission beams. In one embodiment, the demodulated data 112 includes a plurality of beam ensemble data sets corresponding to each of the plurality of transmit beam directions 142, 144, 146. A beam ensemble data set is a combined response to a plurality of transmit beams in a corresponding transmit beam direction. Further, each of the plurality of beam ensemble data sets includes a plurality of echo signal data sets corresponding to each of the plurality of transmission beams 116. An echo signal data set is generated in response to a corresponding transmission beam. In some embodiments, the plurality of echo signal data sets is generated corresponding to the plurality of transmit focal distance values.

The DPU 104 is communicatively coupled to the system front-end unit 102 and configured to acquire the demodulated data 112 and store the demodulated data 112 in the memory buffer unit 132. In one embodiment, the DPU 104 is configured to store the demodulated data 112 in the memory buffer unit 132 as four-dimensional (4D) data. The 4D data includes a plurality of three-dimensional (3D) scanned data indexed by the plurality of transmit beam directions 142, 144, 146. The 3D scanned data in turn includes a plurality of two-dimensional (2D) scanned data indexed by the plurality of transmission beams 116. In one embodiment, the 2D scanned data includes a plurality of complex data samples indexed by a range index and a channel index. The plurality of complex data samples corresponds to the plurality of spatial locations 140. The range index is representative of a spatial depth value and the channel index is representative of a spatial width value corresponding to a spatial location 140. Further, each of the plurality of complex data samples includes a first sample corresponding to an in-phase component value represented by I and a second sample corresponding to a quadrature-phase component value represented by [symbol] . It may be noted that complex data samples corresponding to the plurality of spatial locations 140 may be stored in a plurality of formats in a memory buffer unit 132.

In another embodiment, the 2D scanned data includes a plurality of real-valued, modulated radio frequency (RF) data samples indexed by a range index and a channel index. It may be noted that real-valued RF data samples corresponds to complex data samples in the I-Q format. It may be noted that although the real-valued RF data samples are equivalent to the complex data samples in the I-Q format, the real-valued RF data samples require more memory for storage. In certain embodiments, the 2D scanned data is representative of dynamic image data corresponding to a plurality of time instants during the scanning procedure. In one embodiment, the dynamic image data may be stored in a grid corresponding to a single-line acquisition technique. In another embodiment, the dynamic image data may be stored in a grid corresponding to a multiple-line acquisition technique.

Further, the DPU 104 is configured to obtain a plurality of wave-number vectors 148 and location response data corresponding to each spatial location 140. Each of the plurality of wave-number vectors 148 corresponds to a spatial location 140 and a beam direction 142, 144, 146. The term “wave-number vector” corresponding to a spatial location and a transmission beam direction refers to a unit vector normal to a wave front at the spatial location generated by a beam transmitted along the transmission beam direction.

In one embodiment, the plurality of wave-number vectors 148 is pre-computed for the plurality of transmit beam directions 142, 144, 146 at each of the plurality of spatial locations 140. Furthermore, the color flow scan sequence is used to pre-compute the plurality of wave-number vectors 148. In one embodiment, the plurality of wave-number vectors 148 may be stored in the memory buffer unit 132 and retrieved by the CPU 134. By way of a non-limiting example, the wave-number vectors 148 may be stored in a look-up table in the memory buffer unit 132. In another embodiment, the plurality of wave-number vectors 148 is computed in real-time during processing of the scanned data. In such an embodiment, the computation of the wave-number vectors 148 may be performed by the CPU 134 or the GPU 130 at a first time instant and stored in the memory buffer unit 132. Further, the CPU 134 may retrieve the stored wave-number vectors 148 at a second-time instant that is later than the first time instant. The computation of the wave-number vectors 148 will be described in greater detail with reference to the DPU 104.

Moreover, in one embodiment, the location response data includes a subset of the plurality of beam ensemble data sets corresponding to a subset of the plurality of transmit beam directions. The subset of the plurality of beam ensemble data sets is referred to herein as ‘ensemble subset’ and the subset of the plurality of transmit beam directions is referred to herein as ‘beam direction subset.’ The GPU 130 is also configured to receive the ensemble subset and the beam direction subset from the memory buffer unit 132. In one embodiment, the beam direction subset for a spatial location 140 may be determined based on the geometry of the system front-end unit 102 and the color flow scan sequence. The determination of the ensemble subset and the beam direction subset may be performed off-line and may be stored in the memory buffer unit 132.

Within the DPU 104, the CPU 134 and GPU 130 are communicatively coupled to the memory buffer unit 132 and configured to determine a plurality of Doppler frequency values based on the location response data. In one embodiment, the GPU 130 is configured to determine a beamformed data set corresponding to the ensemble subset. The beamformed data set includes a plurality of beamformed outputs corresponding to transmission beams transmitted along a transmit beam direction selected from the beam direction subset. The plurality of beamformed outputs is generated by combining echo signal data sets corresponding to the ensemble subset.

Further, the DPU 104 is configured to determine a plurality of phase shift values based on the plurality of beamformed outputs. In one embodiment, an autocorrelation based technique is used to determine a phase shift value from a beamformed output among the plurality of beamformed outputs. In another embodiment, a frequency domain approach may be used to determine the phase shift value. The DPU 104 is further configured to determine a mean phase shift value based on the plurality of phase shift values via use of an averaging operation.

Moreover, the DPU 104 is configured to determine a Doppler frequency value as a ratio of the mean phase shift value and the PRI specified by the color flow scan sequence. In one embodiment, the plurality of beamformed outputs may be processed by the DPU 104 using a clutter suppression filter (e.g. a high-pass filter) to generate a plurality of clutter free beamformed outputs. The plurality of clutter free beamformed outputs is used as pixels in an ultrasound image. In certain embodiments, the plurality of clutter free beamformed outputs may be further processed by the DPU 104 using a smoothing filter to generate a plurality of smoothed beamformed outputs. In one embodiment, the DPU 104 is configured to process a plurality of pixels corresponding to the plurality of spatial locations 140 to generate the plurality of smoothed beamformed outputs. In another embodiment, the DPU 104 is configured to process a plurality of pixel values corresponding to the plurality of pixels to generate the plurality of smoothed beamformed outputs. In one example, the clutter free beamformed outputs may be used to determine the Doppler frequency value. In another example, the plurality of smoothed beamformed outputs is processed to determine the Doppler frequency value. Similarly, a plurality of

Doppler frequency values is determined corresponding to the plurality of transmit beam directions based on corresponding plurality of beamformed outputs.

In another embodiment, the DPU 104 is configured to obtain a spatial lag value corresponding to each spatial location based on the ensemble dataset. Specifically, the spatial lag value is determined based on an axial cross correlation technique. In one embodiment, the axial cross correlation technique includes determining a 2D cross correlation corresponding to each spatial location based on the real valued RF data samples. Moreover, a maximum value of the 2D cross correlation is determined. A spatial lag value corresponding to the maximum value is determined as the spatial lag value. Further, an average of a plurality of spatial lag values of spatial locations corresponding to a given transmit beam is determined as an averaged spatial lag value. The averaged spatial lag value is representative of an axial flow velocity corresponding to the given transmit beam. It may be further noted that the spatial lag value is equivalent to a phase shift value corresponding to each spatial location and the averaged spatial lag value is related to the Doppler frequency value at the corresponding location by a scaling factor.

Further, the DPU 104 is configured to determine a plurality of axial flow velocity values corresponding to the plurality of transmit beam directions. Determining the plurality of axial flow velocity values includes determining a plurality of spatial lag values based on the ensemble subset using a cross-correlation technique. In addition, determining the plurality of axial flow velocity values includes determining a mean spatial lag value based on the plurality of spatial lag values. Further, determining the plurality of axial flow velocity values includes computing a ratio of the mean spatial lag value and a PRI corresponding to the color flow scan sequence to generate an axial flow velocity value. It may be noted that the plurality of axial flow velocity values corresponding to the plurality of transmit beam directions may be used instead of the plurality of Doppler frequency values in equation (1) to determine the flow velocity vector.

In one embodiment, the DPU 104 may be configured to determine the plurality of wave-number vectors 148 corresponding to a spatial location 140 and the plurality of transmission beams 116. The DPU 104 is configured to determine a beam direction subset for each spatial location 140. It may be noted that the beam direction subset includes a plurality of relevant transmit beam directions corresponding to each spatial location 140. A transmit beam direction is considered as relevant for a spatial location 140 if a transmission beam transmitted along that transmit beam direction generates one or more echo signals corresponding to that spatial location 140.

Further, the DPU 104 is configured to identify a receiver subarray corresponding to each transmit beam direction among the subset of relevant transmit beam directions. The receiver subarray refers to a plurality of transducer elements in the array of transducer elements 106 receiving response signals generated at the spatial location by a transmission beam transmitted along a specified transmit beam direction. In one example, the DPU 104 is configured to identify a plurality of transducer elements receiving response signals having similar times of arrival (TOAs) from a spatial location. The term ‘time of arrival (TOA)’ as used herein refers to the time for sound to propagate from a spatial location 140 to a transducer element in the array of transducer elements 106. The receiver subarray includes the plurality of such transducer elements. The response signals corresponding to the spatial location are generated in response to a wave front generated along a transmit beam direction. In one embodiment, TOAs in a range from about −5% to about 5% around an average TOA value is considered for identifying the plurality of transducer elements in the receiver subarray. A wave-number vector is determined as a line joining a center of the receiver subarray with the spatial location. In one embodiment, the center of the receiver subarray is identified based on the average TOA value. In another embodiment, the center of the receiver subarray is determined based on a mid-point of the receiver subarray. In one embodiment, the receiver subarray may include all transducer elements 106.

The DPU 104 is further configured to determine a flow vector for each spatial location 140 based on the plurality of Doppler frequency values and the plurality of wave-number vectors. The flow vector at a spatial location 140 in the region of interest 138 is representative of a magnitude and direction of blood flow at the spatial location 140. It may be noted that a Doppler frequency value is a projection of the flow vector on a corresponding wave-number vector at a spatial location. In one embodiment, a matrix equation relating the plurality of Doppler frequency values and the plurality of wave-number vectors is represented as:

$$[ \begin{matrix}{μ}_{1} \\ ⋮ \\ {μ}_{N}\end{matrix} ] = [ \begin{matrix}{w}_{1}^{x} & {w}_{1}^{y} \\ ⋮ & ⋮ \\ {w}_{N}^{x} & {w}_{N}^{y}\end{matrix} ] [ \begin{matrix}{v}_{x} \\ {v}_{y}\end{matrix} ] \qquad ( 1 )$$

In equation (1), μ₁ is a Doppler frequency corresponding to a first transmit beam direction, μ_{N} is a Doppler frequency corresponding to the N^{th} transmit beam direction, vₓ is a first co-ordinate component of a flow vector, v_{y} is a second co-ordinate component of the flow vector, w₁^{x}, w₁^{y} are co-ordinates of a first wave-number vector corresponding to the first transmit beam direction, and w_{N}^{x}, w_{N}^{y} are co-ordinates of the N^{th} wave-number vector corresponding to the Nth transmit beam direction. In another embodiment, μ₁ may represent an axial flow velocity corresponding to the first transmit beam direction and μ_{N} may represent an axial flow velocity corresponding to the N^{th} transmit beam direction.

In one embodiment, the flow vector is estimated by determining a least-squares solution of equation (1). The DPU 104 is configured to determine the flow vector as a least-squares estimate of a mapping of the plurality of Doppler frequency values.

Further, the DPU 104 is also configured to determine a plurality of flow vectors corresponding to the other spatial locations 140. The DPU 104 is also configured to generate a flow vector image 114 based on the plurality of flow vectors corresponding to the plurality of spatial locations 140 within the region of interest 138. In one embodiment, the DPU 104 is further configured to perform a Doppler angle correction operation on each of the plurality of Doppler frequency values based on flow data across the plurality of transmit beam directions. The Doppler angle correction provides an improved estimate of the flow vector magnitude.

In certain embodiments, the DPU 104 may be configured to perform the Doppler angle correction by re-projecting each of the plurality of Doppler frequency value onto a unit vector in a direction of the corresponding flow vector to generate a plurality of corrected Doppler frequency values. The plurality of corrected Doppler frequency values corresponds to the plurality of transmit beam directions. In particular, the DPU 104 is configured to determine a Doppler angle between a wave-number vector and the flow vector corresponding to a spatial location 140. A cosine of the Doppler angle is used to scale the corresponding Doppler frequency value to generate a corrected Doppler frequency value. Furthermore, a corrected mean Doppler frequency value corresponding to the spatial location 140 is determined by averaging the plurality of corrected Doppler frequency values. Also, a corrected flow vector at the spatial location 140 is determined based on the corrected mean Doppler frequency value.

In another embodiment, the DPU 104 may be configured to perform the Doppler angle correction by re-projecting each of a plurality of mean phase shift values corresponding to the plurality of Doppler frequency values onto a corresponding flow vector to generate a plurality of corrected phase shift values. The plurality of corrected phase shift values corresponds to the plurality of transmit beam directions. A plurality of Doppler angles corresponding to the plurality of wave-number vectors is used to correct the plurality of phase shift values. Moreover, an average value of the plurality of corrected phase shift values is computed to determine a corrected mean phase shift value. The corrected mean phase shift value is scaled by the PM to determine a corrected mean Doppler frequency value corresponding to the spatial location 140. The corrected flow vector corresponding to the spatial location 140 is determined based on the corrected mean Doppler frequency value.

Additionally, in some embodiments, the DPU 104 may be configured to perform a correction operation on the plurality of axial flow velocity values across the plurality of transmit directions in the subset of the plurality of transmit beam directions. In one embodiment, the plurality of mean spatial lag values is used for the correction operation. Specifically, the plurality of mean spatial lag values is re-projected onto a corresponding flow vector to generate a plurality of corrected spatial lag values. Further, a corrected flow vector is generated based on an average value of the plurality of corrected spatial lag values.

In one embodiment, the memory buffer unit 132 includes a non-transitory computer readable medium having instructions to enable at least one processing unit to generate the flow vector image 114. Specifically, the instructions enable the at least one processing unit to transmit, using a transmitter array, a set of transmission beams through a region of interest including a plurality of spatial locations. The set of transmission beams includes a plurality of transmission beams corresponding to each of a plurality of transmit beam directions selected from a color flow scan sequence. The instructions further enable the at least one processing unit to generate, using a receiver array, demodulated data in response to the set of transmission beams. The demodulated data includes a plurality of beam ensemble data sets corresponding to each of the plurality of transmit beam directions. Each of the plurality of beam ensemble data sets includes a plurality of echo signal data sets corresponding to each of the plurality of transmission beams. The instructions also enable the at least one processing unit to obtain a plurality of wave-number vectors and location response data corresponding to each spatial location. The location response data includes a subset of the plurality of beam ensemble data sets corresponding to a subset of the plurality of transmit beam directions. The instructions further enable the at least one processing unit to determine a plurality of Doppler frequency values based on the location response data. The instructions enable the at least one processing unit to determine a flow vector for each spatial location based on the plurality of Doppler frequency values and the plurality of wave-number vectors. The instructions also enable the at least one processing unit to generate a flow vector image based on the flow vectors corresponding to the plurality of spatial locations within the region of interest. The flow vector image is representative of a magnitude and direction of blood flow in the region of interest.

The display device 108 is communicatively coupled to the DPU 104 and configured to display at least the flow vector image 114. Each pixel of the flow vector image 114 includes a magnitude and a direction of motion in the region of interest 138. The magnitude is representative of a velocity of blood flow and a direction is representative of a direction of blood flow in the region of interest 138. In one embodiment, the direction is represented by a color value. For example, higher luminance values may represent higher velocities of blood flow. Also, a blue color may be used to represent a blood flow direction away from a user such as a clinician, and a red color may be used to represent a blood flow direction towards the user.

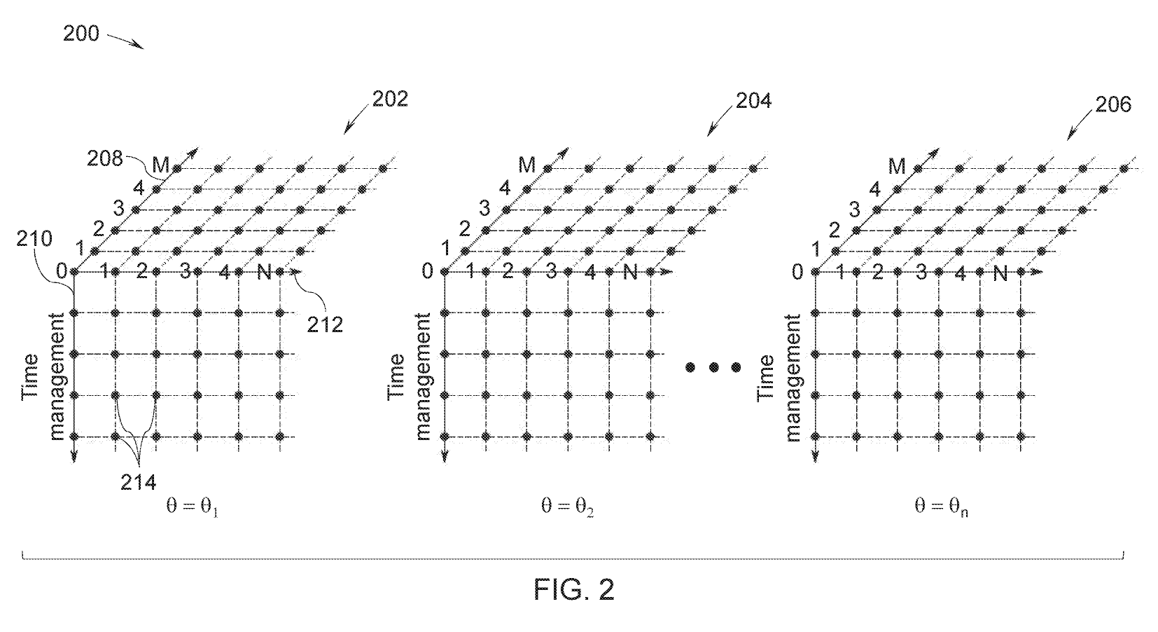

FIG. 2 is a schematic 200 of one example of 4D scanned data stored in memory, in accordance with aspects of the present specification. The schematic 200 illustrates a plurality of 3D data sets 202, 204, 206 indexed by a plurality of transmit beam directions. A number of transmit beam directions used in a scanning procedure is represented by N. Although, only three transmit beam directions are shown in the example of FIG. 2, more than a hundred transmit beam directions may be used in a typical scanning procedure.

Each of the plurality of 3D data sets 202, 204, 206 includes a plurality of 2D data sets 208 indexed by transmission beams transmitted along the corresponding transmit beam direction. The number of transmission beams for each of the transmit beam directions is represented by M. In one embodiment, number of transmission beams M may have a value of five. In another embodiment, number of transmission beams M may have a value of 8. Similarly, other examples may use different values of M.

Each of the plurality of 2D data sets 208 includes a plurality of pixels 214 arranged in a 2D array indexed by a plurality of range values 210 and a plurality of channel numbers 212. The range index is represented by r and channel index is represented by k. The range index r may have a value in a range from 1 to R and the channel index k may have a value in a range from 1 to K. In one embodiment, the range index r represents two hundred fifty six discrete range values (R=256) and the channel index k represents one hundred and twenty eight channel numbers (K=128). It may be noted that 4D data generated by a scanning procedure with M=5, N=100, R=256 and one hundred transducer elements require about 10 MB of memory for storage.

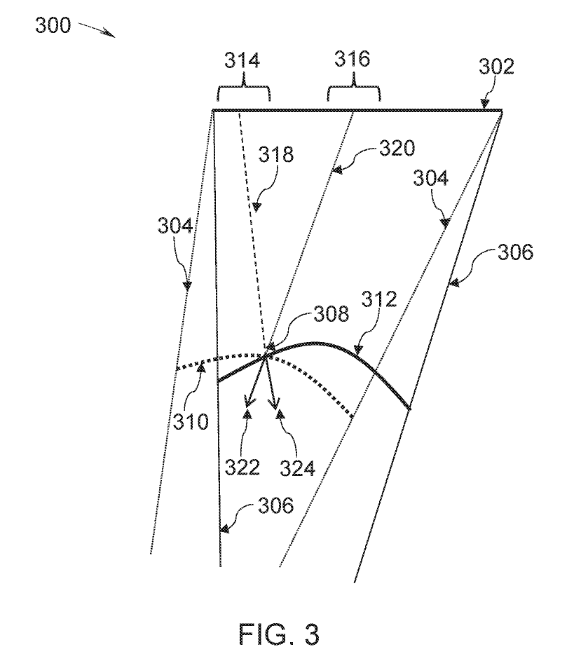

Turning now to FIG. 3, a schematic 300 illustrating determination of wave-number vectors at a spatial location, in accordance with aspects of the present specification, is depicted. The schematic 300 depicts a transducer array 302 and two transmission beams 304 and 306 transmitted along two transmit beam directions. Reference numeral 308 is representative of a spatial location. A first wave front 310 corresponding to a first transmission beam 304 and a second wave front 312 corresponding to a second transmission beam 306 are also depicted in the schematic 300. The two wave fronts 310, 312 that cross the spatial location 308 are identified. The schematic 300 also depicts a first receiving subarray 314 corresponding to the second transmission beam 306 and a second receiving subarray 316 corresponding to the first transmission beam 304. A first line 318 connecting the center of the first receiver subarray 314 and the spatial location 308 determines a first wave-number vector 324. A second line 320 connecting the center of the second receiver subarray 316 and the spatial location 308 determines a second wave-number number vector 322. In the illustrated embodiment, the wave-number vectors 322, 324 are unit vectors.

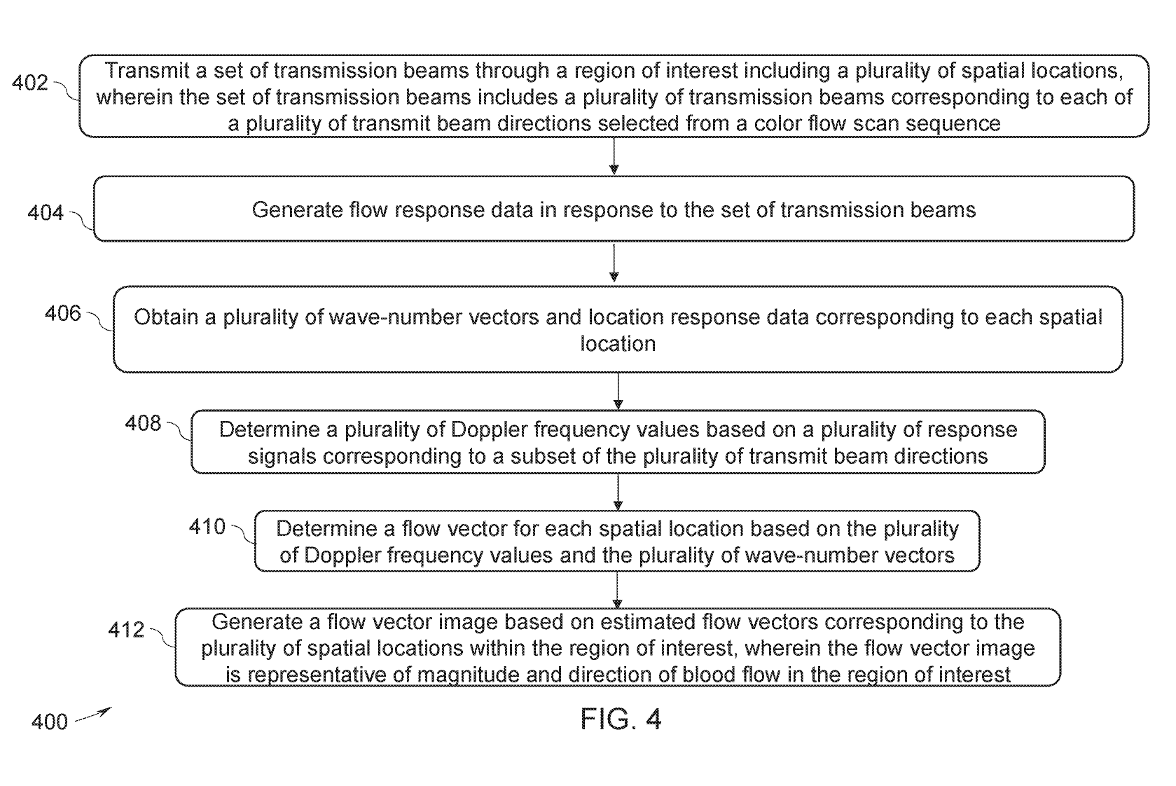

FIG. 4 is a flow chart of a method 400 for ultrasound flow imaging, in accordance with aspects of the present specification. The method 400 includes transmitting, by a transmitter array, a set of transmission beams through a region of interest that includes a plurality of spatial locations, as illustrated in step 402. The set of transmission beams includes a plurality of transmission beams corresponding to each of a plurality of transmit beam directions selected from a color flow scan sequence. In one embodiment, the set of transmission beams includes N transmission beams. One set of transmission beams is transmitted along each of a plurality of transmit beam directions. Each set of transmission beams includes M transmission beams transmitted in quick succession with a time separation specified by the PRI parameter. The parameters N, M and PRI are specified by the color flow scan sequence. In one embodiment, about one hundred transmit beam directions and three to sixteen transmit firings along each transmit beam direction are employed during the scanning. As an example, a PRI parameter in a range from about 62.5 microseconds to about 128 microseconds may be used in the scanning. The plurality of transmission beams may also be referred to as a ‘packet’ and each of the plurality of transmission beams may be referred to as a ‘firing.’ It may be noted that blood flows with different velocities in different types of blood vessels. One example of blood flow is in a range from about 0.03 cm/s to about 40 cm/s.

Further, as indicated by step 404, the method 400 includes generating by a receiver array, demodulated data in response to the set of transmission beams. The demodulated data includes a plurality of beam ensemble data sets corresponding to each of the plurality of transmit beam directions. Each of the plurality of beam ensemble data sets includes a plurality of echo signal data sets corresponding to each of the plurality of transmission beams. A beam ensemble data set is a combined response to a plurality of transmission beams in each transmit beam direction. An echo signal data set is generated in response to a transmission beam.

Subsequently, at step 406, a plurality of wave-number vectors and location response data corresponding to each spatial location are obtained. In one embodiment, the location response data includes a subset of the plurality of beam ensemble data sets (referred herein as ensemble subset) corresponding to a subset of the plurality of transmit beam directions (referred herein as beam direction subset). The beam direction subset of the plurality of transmit beam directions includes one or more transmit beam directions relevant for generating response signals from the spatial location. The response signals of the ensemble subset are used for generating a beamformed output corresponding to the spatial location. The plurality of wave-number vectors is determined corresponding to the plurality of transmit beam directions and a spatial location. For each transmit beam direction a receiver subarray is identified. In one embodiment, the receiver subarray is identified based on TOAs of an echo signal data set corresponding to each spatial location and each transmit beam direction. A line joining the center of the receiver subarray with the spatial location is used to determine a wave-number vector corresponding to the spatial location and the transmit beam direction.

At step 408, a plurality of Doppler frequency values is determined based on the location response data. Specifically, one Doppler frequency value is determined for one transmit beam direction in the beam direction subset. A plurality of beamformed outputs is generated by combining each of the plurality of echo signal data sets. A phase shift value may be determined based on the beamformed signal using an autocorrelation technique. A plurality of phase shift values corresponding to the plurality of transmission beams is determined in a similar manner. A mean phase shift value is determined by averaging the plurality of phase shift values. The Doppler frequency value corresponding the specified transmit beam direction is determined as a ratio of the mean phase shift value and the pulse repetition interval.

Further, a flow vector for each spatial location is determined based on the plurality of Doppler frequency values and the plurality of wave-number vectors, as depicted by step 410. In one embodiment, a flow vector is determined based on the plurality of wave-number vectors and the plurality of Doppler frequency values. By way of example, in some embodiments, equation (1) may be used to determine the flow vectors. In particular, equation (1) relates each wave-number vector to a corresponding Doppler frequency value as a projection of a flow vector. A least-squares solution of the matrix of equation (1) provides an estimate of the flow vector. A plurality of flow vectors corresponding to the plurality of spatial locations may be determined in a similar fashion.

Moreover, at step 412, a flow vector image is generated based on the estimated flow vectors corresponding to the plurality of spatial locations within the region of interest. In one embodiment, the direction of the flow vector is represented with a color value. In one example, a first direction moving away from a viewer may be represented by a blue color and a second direction moving towards the viewer may be represented by a red color. In another embodiment, the magnitude of the flow vector, representative of a velocity of fluid motion, may be represented by grey values.

Systems and method for flow imaging as described hereinabove are configured to determine a flow vector image representative of a magnitude and direction of blood flow in the region of interest using a standard ultrasound image dataset. These systems and method are designed to co-exist with the conventional vector flow imaging techniques in the ultrasound systems. In some embodiments, the systems and methods for flow imaging may be enabled by an operator by selecting a user interface option or may be an automated technique. Furthermore, it may be noted that the robust systems and methods for flow imaging are not dependent on an angle between an ultrasound beam direction and flow direction in the vasculature. and provide freedom to the sonographers from the need to interpret the color flow image for flow directions. Additionally, the systems and methods for flow imaging are suitable for arrays having a smaller foot-print and does not require ultrafast, broad beam acquisition to cover the field of view. The flow vector image generated by the systems and methods for flow imaging overcomes ambiguity of flow direction at spatial locations, thereby enabling easy interpretation of blood flow in the region of interest.

It is to be understood that not necessarily all such objects or advantages described above may be achieved in accordance with any particular embodiment. Thus, for example, those skilled in the art will recognize that the systems and techniques described herein may be embodied or carried out in a manner that achieves or improves one advantage or group of advantages as taught herein without necessarily achieving other objects or advantages as may be taught or suggested herein.

While the technology has been described in detail in connection with only a limited number of embodiments, it should be readily understood that the specification is not limited to such disclosed embodiments. Rather, the technology can be modified to incorporate any number of variations, alterations, substitutions or equivalent arrangements not heretofore described, but which are commensurate with the spirit and scope of the claims. Additionally, while various embodiments of the technology have been described, it is to be understood that aspects of the specification may include only some of the described embodiments. Accordingly, the specification is not to be seen as limited by the foregoing description, but is only limited by the scope of the appended claims.

1. A method for ultrasound flow imaging, comprising:
transmitting, by a transmitter array, a set of transmission beams through a region of interest comprising a plurality of spatial locations, wherein the set of transmission beams comprises a plurality of transmission beams corresponding to each of a plurality of transmit beam directions selected from a color flow scan sequence;
generating, by a receiver array, demodulated data in response to the set of transmission beams, wherein the demodulated data comprises a plurality of beam ensemble data sets corresponding to each of the plurality of transmit beam directions, and wherein each of the plurality of beam ensemble data sets comprises a plurality of echo signal data sets corresponding to each of the plurality of transmission beams;
obtaining a plurality of wave-number vectors and location response data corresponding to each spatial location, wherein the location response data comprises a subset of the plurality of beam ensemble data sets corresponding to a subset of the plurality of transmit beam directions;
determining a plurality of Doppler frequency values based on the location response data;
determining a flow vector for each spatial location based on the plurality of Doppler frequency values and the plurality of wave-number vectors; and
generating a flow vector image based on the flow vectors corresponding to the plurality of spatial locations within the region of interest, wherein the flow vector image is representative of a magnitude and direction of blood flow in the region of interest.

2. The method of claim 1, wherein determining the plurality of Doppler frequency values comprises generating a beamformed data set corresponding to the subset of the plurality of beam ensemble data sets, and wherein the beamformed data set comprises a plurality of beamformed outputs generated by combining echo signal data sets corresponding to the subset of the plurality of beam ensemble data sets.

3. The method of claim 2, wherein determining the plurality of Doppler frequency values comprises:
determining a plurality of phase shift values based on the plurality of beamformed outputs using an autocorrelation technique;
generating a mean phase shift value based on the plurality of phase shift values; and
computing a ratio of the mean phase shift value and a pulse repetition interval corresponding to the color flow scan sequence to generate a Doppler frequency value.

4. The method of claim 2, further comprising processing the plurality of beamformed outputs by a high-pass filter to generate a plurality of clutter free beamformed outputs.

5. The method of claim 4, further comprising processing the plurality of clutter free beamformed outputs by a smoothing filter to generate a plurality of smoothed beamformed outputs.

6. The method of claim 1, wherein obtaining the plurality of wave-number vectors comprises:
identifying a receiver subarray based on times of arrival of an echo signal data set corresponding to each spatial location and each transmit beam direction; and
determining a wave-number vector among the plurality of wave-number vectors, based on a line connecting a center of the receiver subarray with each spatial location.

7. The method of claim 1, wherein determining the flow vector comprises determining a least-squares estimate of a mapping of the plurality of Doppler frequency values.

8. The method of claim 1, further comprising performing a Doppler angle correction on the plurality of Doppler frequency values across the plurality of transmit directions in the subset of the plurality of transmit beam directions.

9. The method of claim 8, wherein performing the Doppler angle correction comprises:
re-projecting the plurality of Doppler frequency values onto a corresponding flow vector to generate a plurality of corrected Doppler frequency values; and
generating a corrected flow vector based on an average value of the plurality of corrected Doppler frequency values.

10. The method of claim 8, wherein performing the Doppler angle correction comprises:
determining a plurality of mean phase shift values corresponding to the subset of the plurality of transmit beam directions;
re-projecting the plurality of mean phase shift values onto a corresponding flow vector to generate a plurality of corrected phase shift values; and
generating a corrected flow vector based on an average value of the plurality of corrected phase shift values.

11. A system for ultrasound flow imaging, comprising:
a system front-end unit configured to:
transmit a set of transmission beams through a region of interest comprising a plurality of spatial locations, wherein the set of transmission beams comprises a plurality of transmission beams corresponding to each of a plurality of transmit beam directions selected from a color flow scan sequence;
generate demodulated data in response to the set of transmission beams, wherein the demodulated data comprises a plurality of beam ensemble data sets corresponding to each of the plurality of transmit beam directions, and wherein each of the plurality of beam ensemble data sets comprises a plurality of echo signal data sets corresponding to each of the plurality of transmission beams;
a digital processor unit communicatively coupled to the system front-end unit and configured to:
acquire the demodulated data;
obtain a plurality of wave-number vectors and location response data corresponding to each spatial location, wherein the location response data comprises a subset of the plurality of beam ensemble data sets corresponding to a subset of the plurality of transmit beam directions;
determine a plurality of Doppler frequency values based on the location response data;
determine a flow vector for each spatial location based on the plurality of Doppler frequency values and the plurality of wave-number vectors;
generate a flow vector image based on the flow vectors corresponding to the plurality of spatial locations in the region of interest, wherein the flow vector image is representative of a magnitude and direction of blood flow in the region of interest; and
a display device communicatively coupled to the digital processor unit and configured to display the flow vector image.

12. The system of claim 11, wherein the digital processor unit is further configured to generate a beamformed data set corresponding to the subset of the plurality of beam ensemble data sets, and wherein the beamformed data set comprises a plurality of beamformed outputs generated by combining echo signal data sets corresponding to the subset of the plurality of beam ensemble data sets.

13. The system of claim 12, wherein the digital processor unit is further configured to:
determine a plurality of phase shift values based on the plurality of beamformed outputs using an autocorrelation technique;
determine a mean phase shift value based on the plurality of phase shift values; and
determine a Doppler frequency value as a ratio of the mean phase shift value and a pulse repetition interval corresponding to the color flow scan sequence.

14. The system of claim 12, wherein the digital processor unit is further configured to process the plurality of beamformed outputs by a clutter suppression filter to generate a plurality of clutter free beamformed outputs.

15. The system of claim 14, wherein the digital processor unit is further configured to process the plurality of clutter free beamformed outputs by a smoothing filter to generate a plurality of smoothed beamformed outputs.

16. The system of claim 11, wherein the digital processor unit is further configured to:
identify a receiving subarray based on times of arrival of an echo signal data set corresponding to each spatial location and each transmit beam direction; and
determine a wave-number vector among the plurality of wave-number vectors, based on a line connecting a center of the receiver subarray with each spatial location.

17. The system of claim 11, wherein the digital processor unit is further configured to determine a least-squares estimate of a mapping of the plurality of Doppler frequency values.

18. The system of claim 11, wherein the digital processor unit is further configured to perform a correction operation on the plurality of Doppler frequency values across the plurality of transmit directions in the subset of the plurality of transmit beam directions.

19. The system of claim 18, wherein, to perform the correction operation on the plurality of Doppler frequency values, the digital processor unit is configured to:
re-project the plurality of Doppler frequency values onto a corresponding flow vector to generate a plurality of corrected Doppler frequency values; and
generate a corrected flow vector based on an average value of the plurality of corrected Doppler frequency values.

20. The system of claim 18, wherein the digital processor unit is configured to:
determine a plurality of mean phase shift values corresponding to the subset of the plurality of transmit beam directions;
re-project the plurality of mean phase shift values onto a corresponding flow vector to generate a plurality of corrected phase shift values; and
generate a corrected flow vector based on an average value of the plurality of corrected phase shift values.

21. A non-transitory computer readable storage medium for ultrasound flow imaging using a processing unit, the non-transitory computer readable storage medium including instructions to command the processor to:
transmit, by a transmitter array, a set of transmission beams through a region of interest comprising a plurality of spatial locations, wherein the set of transmission beams comprises a plurality of transmission beams corresponding to each of a plurality of transmit beam directions selected from a color flow scan sequence;
generate, by a receiver array, demodulated data in response to the set of transmission beams, wherein the demodulated data comprises a plurality of beam ensemble data sets corresponding to each of the plurality of transmit beam directions, and wherein each of the plurality of beam ensemble data sets comprises a plurality of echo signal data sets corresponding to each of the plurality of transmission beams;
obtain a plurality of wave-number vectors and location response data corresponding to each spatial location, wherein the location response data comprises a subset of the plurality of beam ensemble data sets corresponding to a subset of the plurality of transmit beam directions;
determine a plurality of Doppler frequency values based on the location response data;
determine a flow vector for each spatial location based on the plurality of Doppler frequency values and the plurality of wave-number vectors; and
generate a flow vector image based on the flow vectors corresponding to the plurality of spatial locations within the region of interest, wherein the flow vector image is representative of a magnitude and direction of blood flow in the region of interest.
